(19) United States
(12) Patent Application Publication
(10) Pub. No.: US 2025/0204911 A1
(43) Pub. Date: Jun. 26, 2025

(54) SURGICAL STAPLER HAVING SHAFT RECOGNITION MECHANISM

(71) Applicant: Applied Medical Resources Corporation, Rancho Santa Margarita, CA (US)

(72) Inventors: Stephen Eldridge, San Clemente, CA (US); Timothy M. Hopkins, Rancho Santa Margarita, CA (US); Eric J. Weiss, San Clemente, CA (US); Bruno Vu, Rancho Santa Margarita, CA (US); Jonathan R. Nash, Rancho Santa Margarita, CA (US); Andrew Krasts, San Clemente, CA (US); Isabella Hidalgo, Irvine, CA (US); Anay Pandit, Lake Elsinore, CA (US)

(21) Appl. No.: 19/076,751

(22) Filed: Mar. 11, 2025

Related U.S. Application Data
(63) Continuation of application No. 17/575,986, filed on Jan. 14, 2022, now Pat. No. 12,279,767.
(60) Provisional application No. 63/137,652, filed on Jan. 14, 2021.

(51) Int. Cl. A61B 17/068

(52) U.S. Cl. CPC A61B 17/068

(57) ABSTRACT

A handle assembly for a surgical stapler can comprise a rotatable actuation shaft. The actuation shaft can have a first rotational orientation in which it can actuate a jaw assembly in a staple firing mode, and a second rotational orientation in which it can actuate a jaw assembly in a reversing mode. The handle assembly can include an articulation mechanism including a ball screw to selectively articulate the jaw assembly relative to an elongate shaft. The handle assembly can include a mechanism to lock out selection of the staple firing mode and actuation of the articulation mechanism if a reload shaft is not securely coupled to the handle assembly.

### CROSS-REFERENCE TO RELATED APPLICATIONS

This application is a continuation of U.S. patent application Ser. No. 17/575,986 entitled “Surgical Stapler Having Shaft Recognition Mechanism” filed on Jan. 14, 2022 which claims priority to and benefit of U.S. Provisional Patent Application Ser. No. 63/137,652 entitled “Surgical Stapler Having Shaft Recognition Mechanism” filed on Jan. 14, 2021 each of which is incorporated herein by reference in its entirety.

### BACKGROUND OF THE INVENTION

### Field of the Invention

The present application relates generally to surgical occlusion instruments and, more particularly, to surgical staplers.

### Description of the Related Art

Surgical staplers are used to approximate or clamp tissue and to staple the clamped tissue together. As such, surgical staplers have mechanisms to ensure that tissue is properly positioned and captured and to drive staples through the tissue. As a result, this has produced, for example, multiple triggers and handles in conjunction with complex mechanisms to provide proper stapling of the clamped tissue. With these complex mechanisms, surgical staplers can have increased manufacturing burdens, as well as potential sources for device failure and confusion for the user. Thus, reliable stapling of clamped tissue without complex mechanisms is desired.

### SUMMARY OF THE INVENTION

In certain embodiments, a handle assembly for a surgical stapler is provided herein. The handle assembly comprises a handle body, an actuation shaft, and a shaft coupling firing lockout mechanism. The handle body comprises a stationary handle and a movable handle pivotably coupled to the handle body. The handle body comprises a coupler configured to removably couple to an instrument shaft having a stapler jaw assembly. The actuation shaft is mechanically coupled to the movable handle for manual actuation thereof. The actuation shaft is slidable within the handle body along a longitudinal axis. The actuation shaft is selectively positionable in a first orientation wherein movement of the movable handle relative to the stationary handle distally advances the actuation shaft and a second orientation wherein movement of the movable handle relative to the stationary handle proximally retracts the actuation shaft. The shaft coupling firing lockout mechanism prevents selective positioning of the actuation shaft in the first orientation when no instrument shaft is coupled to the coupler and allows selective positioning of the actuation shaft in the first orientation when the instrument shaft is coupled to the coupler.

In certain embodiments, a handle assembly for a surgical stapler is provided. The handle assembly comprises a handle body, an actuation shaft, an articulation mechanism, an articulation lockout mechanism, and a shaft coupling firing lockout mechanism. The handle body comprises a stationary handle and a movable handle pivotably coupled to the handle body. The handle body comprises a coupler configured to removably couple to an instrument shaft having a stapler jaw assembly. The actuation shaft is mechanically coupled to the movable handle for manual actuation thereof. The actuation shaft is slidable within the handle body along a longitudinal axis. The actuation shaft is selectively positionable in a firing orientation and a reverse orientation. The articulation mechanism is configured to articulate the stapler jaw assembly relative to the instrument shaft when the instrument shaft is coupled to the handle body. The articulation lockout mechanism prevents operation of the articulation mechanism when no instrument shaft is coupled to the coupler and allows operation of the articulation mechanism when the instrument shaft is coupled to the coupler. The shaft coupling firing lockout mechanism, the shaft coupling firing lockout mechanism prevents selective positioning of the actuation shaft in the first orientation when no instrument shaft is coupled to the coupler and allows selective positioning of the actuation shaft in the first orientation when the instrument shaft is coupled to the coupler.

In certain embodiments, a handle assembly for a surgical stapler is provided. The handle assembly comprises a handle body, an actuation shaft, a rotation mechanism, and a shaft coupling firing lockout mechanism. The handle body comprises a stationary handle and a movable handle pivotably coupled to the handle body. The handle body having a coupler configured to removably couple to an instrument shaft. The actuation shaft is mechanically coupled to the movable handle for manual actuation thereof. The actuation shaft is slidable within the handle body along a longitudinal axis and rotatable relative to the longitudinal axis to selectively position the actuation shaft in one of a firing orientation and a reverse orientation. The rotation mechanism selectively rotates the actuation shaft. The shaft coupling firing lockout mechanism comprises a lockout sleeve and at least one lockout arm. The lockout sleeve is positioned adjacent the coupler. The at least one lockout arm is coupled to the lockout sleeve and extends proximally from the lockout sleeve. The lockout arm comprises a lockout tab protruding therefrom. The shaft coupling firing lockout mechanism is positioned in a locked out configuration when no instrument shaft is coupled to the coupler such that the lockout tab interferes with operation of the rotation mechanism to position the actuation shaft in a firing orientation. The shaft coupling firing lockout mechanism is positioned in an unlocked configuration when the instrument shaft is coupled to the coupler such that the lockout tab is spaced apart from the rotation mechanism.

In certain embodiments, a surgical stapler is provided. The surgical stapler comprises an instrument shaft and a handle assembly. The instrument shaft has a proximal end and a distal end. The instrument shaft comprises an end effector at the distal end and a lockout keyway at the proximal end. The handle assembly comprises a handle body, an actuation shaft, an articulation mechanism, and an integrated articulation lockout and shaft coupling firing lockout mechanism. The handle body comprises a stationary handle and a movable handle pivotably coupled to the handle body. The handle body has a coupler configured to removably couple to the instrument shaft. The actuation shaft is mechanically coupled to the movable handle for manual actuation thereof. The actuation shaft is slidable within the handle body along a longitudinal axis and selectively positionable in one of a firing orientation and a reverse orientation. The articulation mechanism is operable to articulate the end effector relative to the instrument shaft when the instrument shaft is coupled to the coupler. The integrated mechanism comprises a lockout sleeve positioned adjacent the coupler; at least one lockout arm coupled to the lockout sleeve; and a locking sleeve. The at least one lockout arm extends proximally from the lockout sleeve to a proximal end. The lockout arm comprises a lockout tab protruding therefrom. The locking sleeve is coupled to the proximal end of the at least one lockout arm. The lockout keyway of the shaft is engageable with the lockout sleeve such that integrated mechanism is positioned in a locked out configuration when no instrument shaft is coupled to the coupler such that the lockout tab interferes with positioning the actuation shaft in a firing orientation and the locking sleeve prevents operation of the articulation mechanism. The integrated mechanism is positioned in an unlocked configuration when the instrument shaft is coupled to the coupler such that the lockout tab is positioned to allow positioning of the actuation shaft in the firing orientation and the locking sleeve is positioned to allow operation of the articulation mechanism.

### BRIEF DESCRIPTION OF THE DRAWINGS

FIG. 1 is a perspective view of an embodiment of surgical stapling device with the jaws in an open configuration;

FIG. 2 is a perspective view of an embodiment of a reload shaft for the surgical stapling device of FIG. 1 with the jaws in a closed configuration;

FIG. 3 is a perspective view of an embodiment of a handle assembly having an articulation mechanism for a surgical stapling device;

FIG. 4 is a perspective view of the handle assembly of FIG. 3 with a movable handle in a closed configuration;

FIG. 5 is a top view of the handle assembly of FIG. 3 with a selector in a first configuration;

FIG. 6 is a top view of the handle assembly of FIG. 3 with a selector in a second configuration;

FIG. 7A is a side view of the handle assembly of FIG. 3;

FIG. 7B is a cross-sectional side view of the handle assembly of FIG. 3;

FIG. 8A is a cross-sectional side view of the handle assembly of FIG. 1 in an initial configuration;

FIG. 8B is a cross-sectional perspective view of the handle assembly of FIG. 8A;

FIG. 9A is a cross-sectional side view of the handle assembly of FIG. 1 actuated to a closed configuration;

FIG. 9B is a cross-sectional perspective view of the handle assembly of FIG. 9A;

FIG. 10A is a cross-sectional side view of the handle assembly of FIG. 1 in a forward drive configuration;

FIG. 10B is a cross-sectional perspective view of the handle assembly of FIG. 10A;

FIG. 11A is a cross-sectional side view of the handle assembly of FIG. 1 in the forward drive configuration;

FIG. 11B is a cross-sectional perspective view of the handle assembly of FIG. 11A;

FIG. 12A is a cross-sectional side view of handle assembly of FIG. 1 in a fully driven forward configuration;

FIG. 12B is a cross-sectional perspective view of the handle assembly of FIG. 12A;

FIG. 13A is a cross-sectional side view of the handle assembly of FIG. 1 in a reverse drive configuration;

FIG. 13B is a cross-sectional perspective view of the handle assembly of FIG. 13A;

FIG. 14A is a cross-sectional side view of the handle assembly of FIG. 1 in a fully driven reverse configuration;

FIG. 14B is a cross-sectional perspective view of the handle assembly of FIG. 14A;

FIG. 15 is a cut-away side view of an embodiment of articulation mechanism of the handle assembly of FIG. 3;

FIG. 16 is a perspective view of the articulation mechanism of FIG. 15 in an articulated position;

FIG. 17A is a cut-away side view of the articulation mechanism of FIG. 15;

FIG. 17B is a cut-away side view of the articulation mechanism of FIG. 15 with a release button depressed;

FIG. 17C is a cut-away side view of the articulation mechanism of FIG. 15 with the release button depressed and partially returned to a centered position;

FIG. 17D is a cut-away side view of the articulation mechanism of FIG. 15 with the release button depressed and returned to a centered position;

FIG. 17E is a cut-away side view of the articulation mechanism of FIG. 15 in the centered position with the release button partially released;

FIG. 17F is a cut-away side view of the articulation mechanism of FIG. 15;

FIG. 18A is a perspective view of an articulation lockout mechanism and a shaft coupling firing lockout mechanism of the handle assembly of FIG. 3 in a locked out configuration;

FIG. 18B is a perspective view of an articulation lockout mechanism and a shaft coupling firing lockout mechanism of the handle assembly of FIG. 3 in an unlocked configuration;

FIG. 19A is a perspective view of a shaft coupling firing lockout mechanism of the handle assembly of FIG. 3 in a locked out configuration;

FIG. 19B is a perspective view of a shaft coupling firing lockout mechanism of the handle assembly of FIG. 3 in an unlocked configuration;

FIG. 20A is a cut away top view of an articulation lockout mechanism of the handle assembly of FIG. 3 in a locked out configuration;

FIG. 20B is a cut away top view of an articulation lockout mechanism of the handle assembly of FIG. 3 in an unlocked configuration;

FIG. 21A is a side view of an embodiment of reload shaft for the surgical stapling system of FIG. 1;

FIG. 21B is a perspective end view of a proximal end of the reload shaft of FIG. 21A;

FIG. 21C is a side view of an embodiment of lockout keyway for a reload shaft of FIG. 21A;

FIG. 21D is perspective view of a lockout sleeve of the handle assembly of FIG. 3;

FIG. 21E is a schematic view illustrating a coupling operation of an embodiment of lockout keyway of a reload shaft with a lockout sleeve of a handle assembly;

FIG. 21F is a cut away top view of a shaft coupling firing lockout mechanism of the handle assembly of FIG. 3 in an engagement configuration; and

FIG. 21G is a view of the shaft coupling firing lockout mechanism of FIG. 21F in an unlocked configuration.

### DETAILED DESCRIPTION OF THE INVENTION

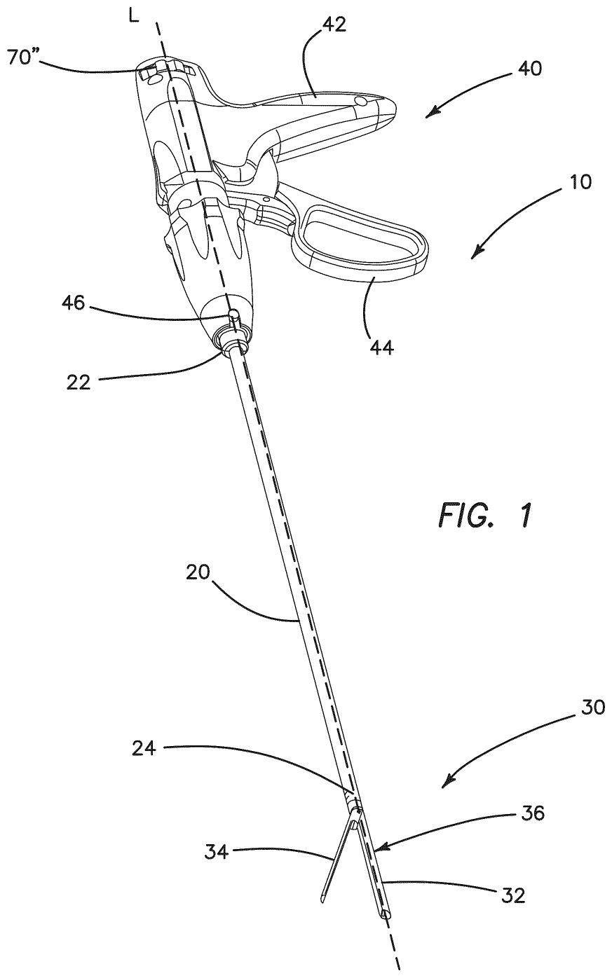

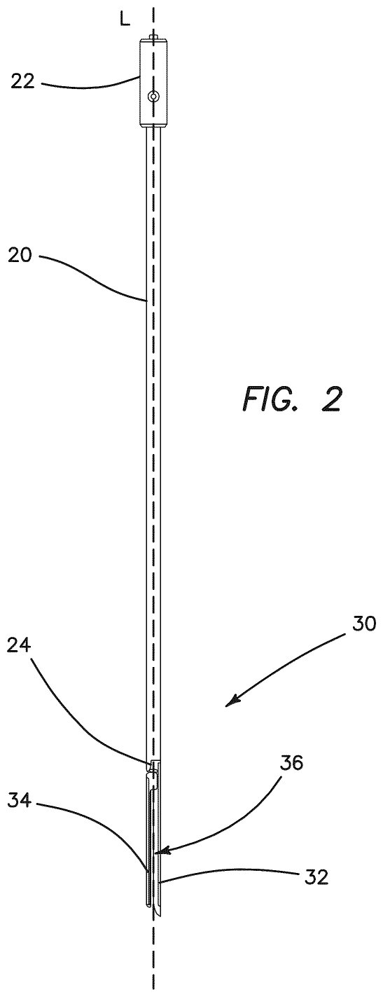

With reference to FIGS. 1-2, an embodiment of surgical stapling device is illustrated. The illustrated embodiment of surgical stapler 10 comprises an elongate shaft 20, a jaw assembly 30, and a handle assembly 40. FIG. 1 illustrates the surgical stapler 10 with the jaw assembly 30 in an open configuration. FIG. 2 illustrates a removable reload shaft assembly comprising the elongate shaft 20 and jaw assembly 30 of the surgical stapler 10 with the jaw assembly 30 in a closed configuration.

With continued reference to FIGS. 1 and 2, the illustrated embodiment of surgical stapler 10 can be sized and configured for use in laparoscopic surgical procedures. For example, the elongate shaft 20 and jaw assembly 30 can be sized and configured to be introduced into a surgical field through an access port or trocar cannula. In some embodiments, the elongate shaft 20 and jaw assembly 30 can be sized and configured to be inserted through a trocar cannula having a relatively small working channel diameter, such as, for example, less than 8 mm. In other embodiments, elongate shaft 20 and jaw assembly 30 can be sized and configured to be inserted through a trocar cannula having a larger working channel diameter, such as, for example, 10 mm, 11 mm, 12 mm, or 15 mm. In other embodiments, it is contemplated that certain aspects of the surgical staplers described herein can be incorporated into a surgical stapling device for use in open surgical procedures.

With continued reference to FIGS. 1 and 2, as illustrated, the elongate shaft 20 comprises a generally tubular member. The elongate shaft 20 extends from a proximal end 22 to a distal end 24. The elongate shaft 20 defines a central longitudinal axis, L. of the surgical stapler 10 extending between the proximal end 22 and the distal end 24.

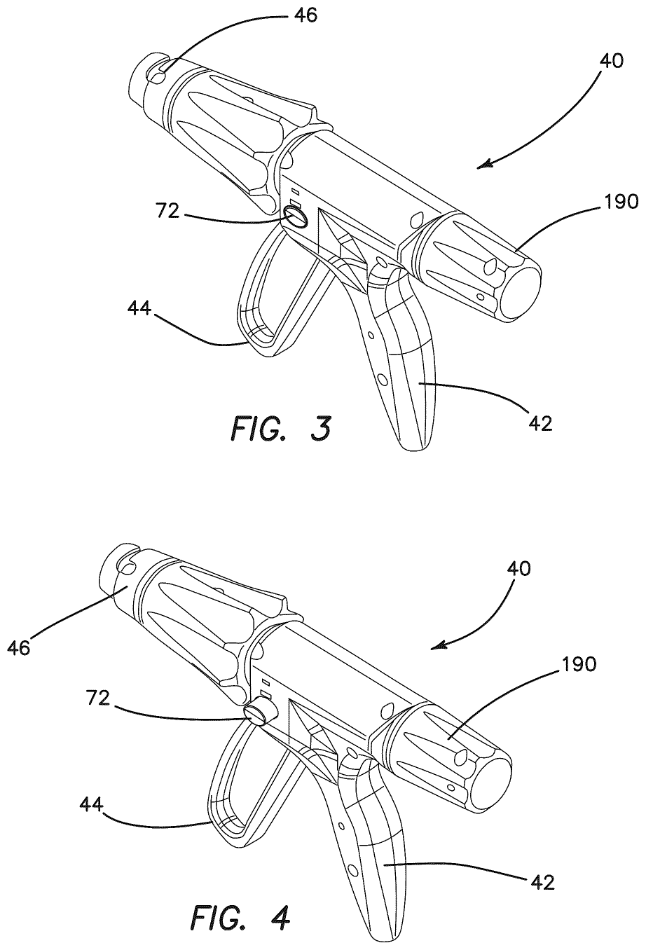

With continued reference to FIGS. 1 and 2, in the illustrated embodiment, the jaw assembly 30 is coupled to the elongate shaft 20 at the distal end 24 of the elongate shaft 20. The jaw assembly 30 comprises a first jaw 32 and a second jaw 34 pivotally coupled to the first jaw 32. In the illustrated embodiment of FIGS. 3 and 15-17, the jaw assembly 30 is articulable with respect to the elongate shaft 20 responsive to an articulation mechanism in the handle. FIG. 1 and FIGS. 8-14 illustrate a handle assembly that are configured to fire a centrally-fixed jaw assembly to further illustrate an actuation mechanism of the handle assembly in a staple firing sequence. In an initial configuration, the first jaw 32 includes a plurality of staples 36 disposed therein.

With continued reference to FIGS. 1 and 2, in the illustrated embodiment, the jaw assembly 30 can be actuated from an open configuration (FIG. 1) to a closed configuration (FIG. 2) to a stapling configuration by an actuation member or beam that is longitudinally slideable within the elongate shaft. In an initial position, the beam can be positioned at the distal end 24 of the elongate shaft 20. With the beam in the initial position, the second jaw 34 is pivoted away from the first jaw 32 such that the jaw assembly 30 is in the open configuration. The actuation beam engages the second jaw 34 upon translation of the actuation member or beam distally along the longitudinal axis L. Translation of the actuation beam distally from the initial position a first distance can actuate the jaw assembly from the open configuration to the closed configuration. With the jaw assembly 30 in the closed configuration, the actuation beam can be returned proximally the first distance to return the jaw assembly 30 to the open configuration. A distal end of the actuation beam can advance a staple slider configured to deploy staples from the first jaw 32 such that further translation of the actuation beam distally past the first distance deploys the plurality of staples 36 from the first jaw 32.

With continued reference to FIGS. 1 and 2, in the illustrated embodiment, the handle assembly is coupled to the elongate shaft 20 at the proximal end 22 of the elongate shaft 20. As illustrated, the handle assembly 40 has a pistol grip configuration with a housing defining a stationary handle 42 and a movable handle 44 or trigger pivotably coupled to the stationary handle 42. It is contemplated that in other embodiments, surgical stapler devices including aspects described herein can have handle assemblies with other configurations such as, for example, scissors-grip configurations, or in-line configurations. As further described in greater detail below, the handle assembly 40 houses an actuation mechanism configured to selectively advance an actuation shaft responsive to movement of the movable handle 44.

In some embodiments, the surgical stapler 10 can include the plurality of staples 36 positioned in a disposable cartridge while the handle assembly 40 is configured to be reused with multiple staple cartridges. In the illustrated embodiment, the elongate shaft 20 and jaw assembly 30 define a disposable cartridge that is removably couplable to the handle assembly 40. Accordingly, in the illustrated embodiment the handle assembly 40 includes a coupler 46 at the distal end thereof. The coupler 46 is adapted to engage the elongate shaft 20 of the surgical stapler 10 The coupler 46 can have a bayonet connection having an outer connector that can removably couple the handle assembly 40 to the elongate shaft 20, and an inner connector that can removably couple the actuation shaft of the handle assembly 42 to the actuation member of the elongate shaft 20. Accordingly, the surgical stapler 10 can be configured such that the handle assembly 40 can be reused with multiple disposable cartridges during a surgical procedure. It is contemplated that in other embodiments, the handle assembly and some portion of the elongate shaft can be reusable while a remainder of the elongate shaft and the jaw assembly define a disposable cartridge. In certain other embodiments, the handle assembly and the elongate shaft can be reusable while the jaw assembly defines a disposable cartridge. In still other embodiments, a jaw insert housing a plurality of staples can define a disposable cartridge while the remainder of the surgical stapler is reusable.

FIGS. 3-7 illustrate various views of an embodiment of handle assembly 40 having an articulation mechanism for a surgical stapler 10. The articulation mechanism can be positioned at the proximal end of the handle assembly 40 and have an articulation knob 190 for articulation of the jaw assembly. In FIG. 3, a perspective view of the handle assembly 40 as illustrated with the movable handle 44 in an open position spaced apart from the stationary handle 42. The illustrated handle assembly 40 further comprises a selector 72 operably coupled to the actuation mechanism housed within the handle assembly 40 as further discussed herein. As illustrated in FIG. 3, the selector 72 is in a first position.

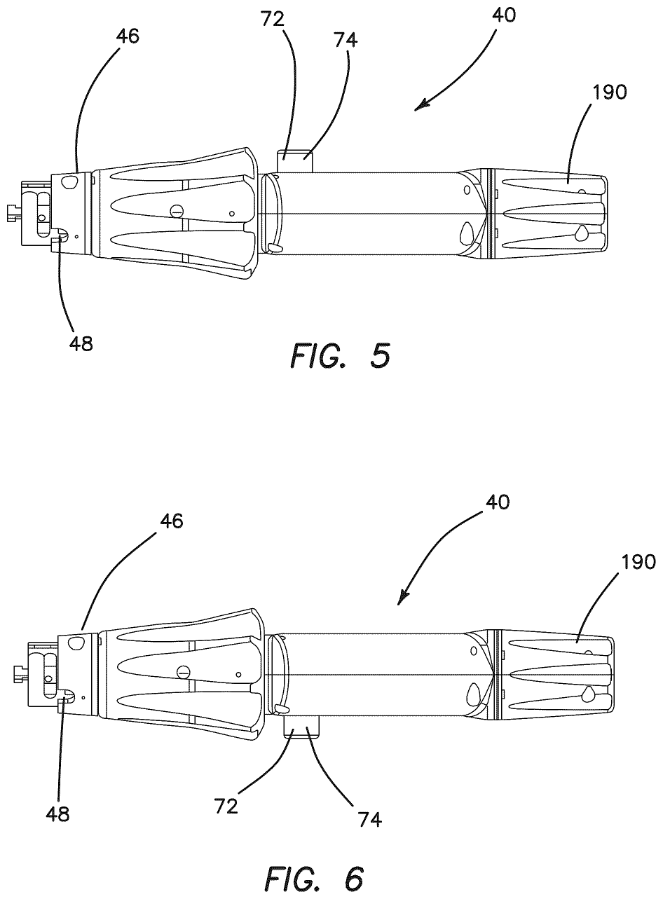

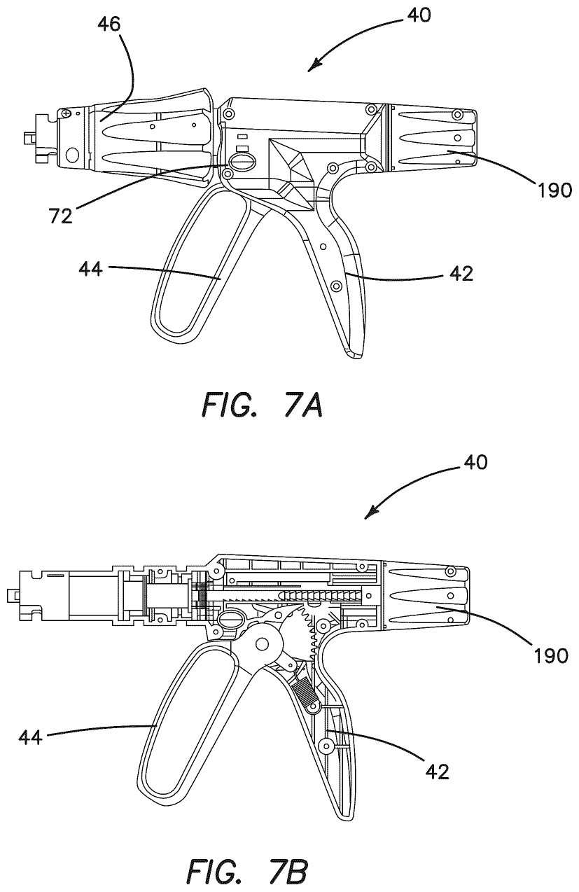

With reference to FIG. 4, another perspective view of the handle assembly 40 of FIG. 3 is illustrated. As illustrated, the movable handle 44 is in the open position positioned adjacent the stationary handle 42, and the selector 72 is in a second position. FIGS. 5 and 6 illustrate a top view of the handle assembly of FIG. 3 with the selector 72, such as a slider 74, in the first position (FIG. 5), and in the second position (FIG. 6). FIG. 7A illustrates a side view of the handle assembly 40 of FIG. 3, and FIG. 7B illustrates a cross-sectional side view of the handle assembly 40 of FIG. 3.

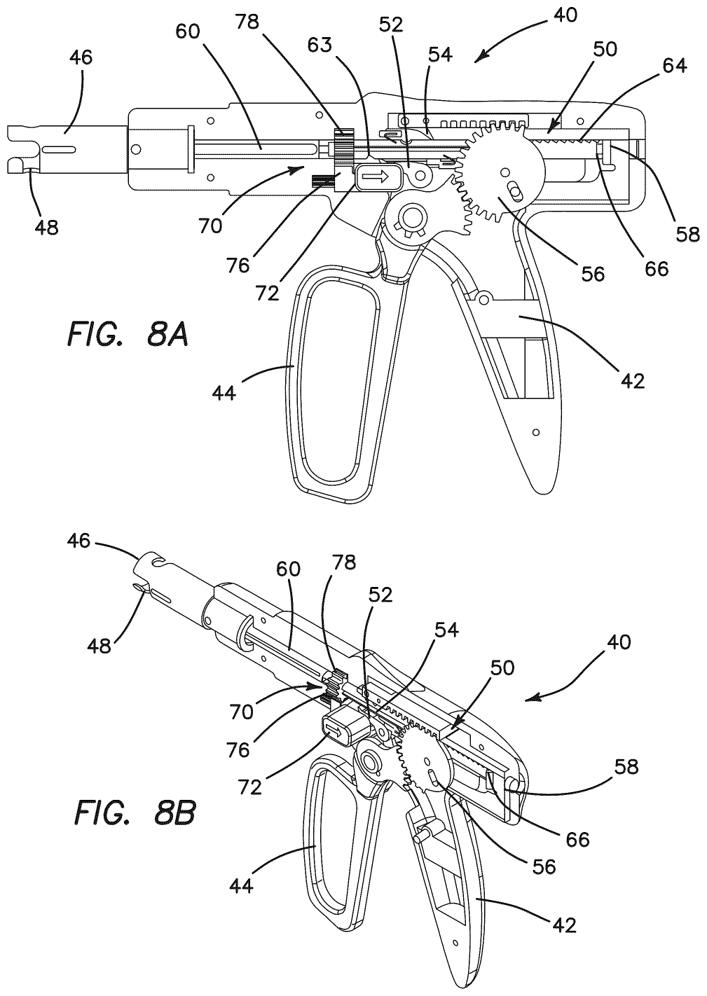

FIGS. 8A and 8B illustrate cross-sectional views of the handle assembly 40 in an initial configuration, revealing operation of the actuation mechanism 50. In the illustrated embodiment, the actuation mechanism 50 is configured to selectively translate the actuation shaft 60 from a first position corresponding to the jaw assembly 30 being in the open configuration to a second position corresponding to the jaw assembly 30 being in the closed configuration and from the second position to a third position to position the jaw assembly 30 in a stapling configuration and deploy the plurality of staples 36. In the initial configuration illustrated in FIGS. 8A and 8B, actuation mechanism 50 can repeatedly translate the actuation shaft 60 between the first position and the second position responsive to movement of the movable handle 44 or trigger without deploying the staples to provide an open and close functionality. This open and close functionality allows a user to position, clamp tissue, and reposition the stapler 10 to find a desirable staple placement location before deploying the staples.

With reference to FIGS. 8-14, in the illustrated embodiment, the actuation mechanism comprises an advancing or forward driver 52, a reverse driver 54, an opening driver 58, an advancing surface 62, a reversing surface 64, and an opening surface 66. The forward driver 52 can be operably coupled to the movable handle 44 such that movement of the movable handle 44 from the open position to the closed position advances the forward driver 52 in a first direction such as for example distally within the handle assembly 40. The forward driver 52 can comprise a pawl or tooth configured to engage a recess or slot.

The reverse driver 54 can be operably coupled to movable handle 44 such that movement of the movable handle 44 from the open position to the closed position advances the reverse driver 54 in a second direction opposite the first direction such as, for example proximally within the handle assembly 40. In some embodiments, the movable handle 44 can be operably coupled with the reverse driver 54 with a geared connection including an idler gear 56. The reverse driver 54 can comprise a pawl or tooth configured to engage a recess or slot.

The opening driver 58 can be operably coupled to the movable handle 44 such that movement of the movable handle 44 from the open position to the closed position advances the opening driver 58 in a first direction such as for example distally within the handle assembly 40. In the illustrated embodiment, the opening driver 58 is coupled to the idler 56 with a pin and slot connection to operably couple the opening driver 58 to the movable handle 44. The opening driver 58 can comprise a pawl or tooth configured to engage a recess or slot.

The actuation shaft 60 includes advancing surface 62, reversing surface 64, and opening surface 66 formed thereon. In the illustrated embodiment, the advancing surface 62 comprises a rack, or plurality of spaced recesses or teeth formed longitudinally along the actuation shaft 60. As illustrated, reversing surface 64 comprises a rack or plurality of space recesses or teeth formed longitudinally along the actuation shaft 60 and angularly offset from the advancing surface 62. In the illustrated embodiment, the opening surface 66 comprises a recess formed in the actuation shaft 60.

In certain embodiments, the actuation shaft 60 is rotatable within the handle assembly 40 about the longitudinal axis of the stapler 10. The handle assembly 40 can comprise a rotation mechanism 70 to provide selective rotation of the actuation shaft 60 within the handle assembly 40. The actuation shaft 60 can be rotatable between a first orientation in which the forward driver 52 is engageable with the advancing surface 62 and a second orientation in which the reverse driver 54 is engageable with the reversing surface 64. With the angular offset of the advancing surface 52 from the reversing surface 54 with respect to the actuation shaft 60, with the actuation shaft in the first orientation, the reverse driver 54 is disengaged from the reversing surface 64, and with the actuation shaft in the second orientation, the forward driver 52 is disengaged from the advancing surface 62.

With continued reference to FIGS. 8-14, in certain embodiments, the rotation mechanism 70 comprises a selector 72, such as a slider. The slider can extend transversely through the housing of the handle assembly 40. The slider can be operably coupled to the actuation shaft 60 such that positioning the slider in the first position extending from one side of the handle assembly 40 positions the actuation shaft 60 in the first orientation, and positioning the slider in the second position extending from an opposite side of the handle assembly 40 rotates the actuation shaft 60 to the second orientation. In the illustrated embodiment, the slider is coupled to a rack 76 in meshing engagement with a gear 78 that is rotatably fixed to the actuation shaft 60 and longitudinally slideable along the actuation shaft 60 (such as, for example, with a keyed connection). Desirably, the illustrated rotation mechanism 70 including a slider discretely positions the actuation shaft 60 in a desired orientation, reducing the incidence of the mismeshed gearing within the actuation mechanism 50. In some embodiments the slider can include visual indicators, such as arrows, to indicate the orientation of the actuation shaft 60, and thus, the actuation mode of the stapler to a user. In other embodiments, the rotation mechanism 70″ (FIG. 1) can include another mechanism such as a rotatable knob directly rotationally coupled to the actuation shaft, rather than the slider selector 72.

In the illustrated embodiment, the advancing surface 62 and the reverse surface 64 are angularly offset by approximately 90 degrees about the actuation shaft. Thus, the rotation mechanism 70 is configured to rotate the actuation shaft approximately 90 degrees between the first orientation and the second orientation. In other embodiments, the actuation surface 62 and the reverse surface 64 can have a different angular offset, such as, for example 120 degrees, and the rotation mechanism 70 can be configured to rotate the actuation shaft 60 correspondingly. Moreover, as described in further detail herein with respect to an open/close mode of the handle assembly 40 operation, in the illustrated embodiment, the opening driver 58 engages with the actuation shaft in the second orientation, in other embodiments, the actuation shaft can be rotatable to a third orientation in which the opening driver 58 engages with the actuation shaft.

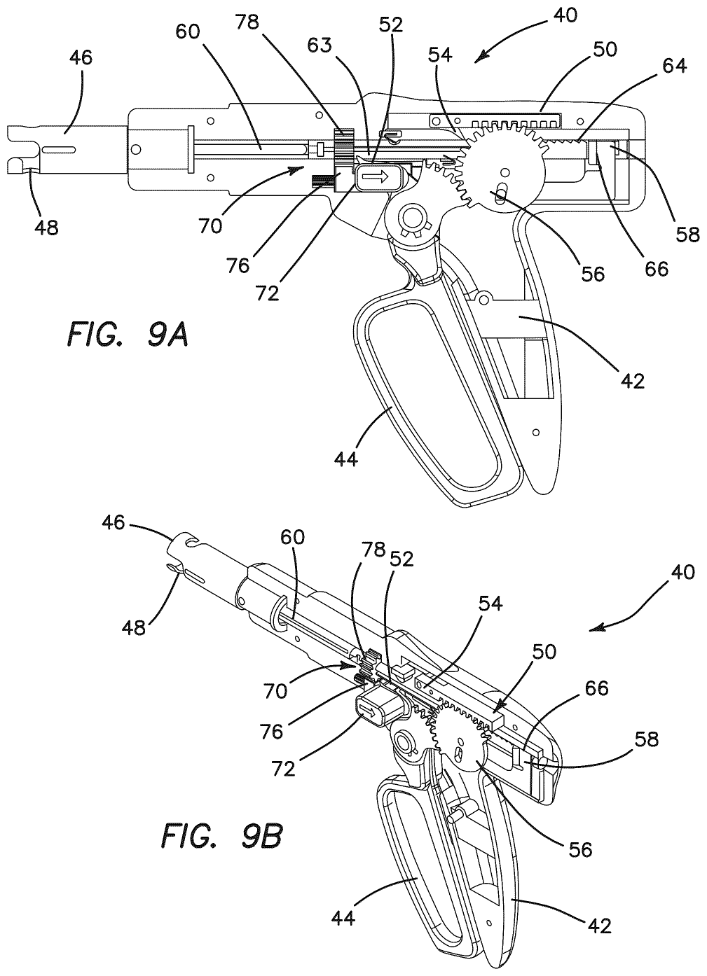

With reference to FIGS. 8-14, a typical operation sequence of the actuation mechanism 50 of the handle assembly 40 is illustrated. FIGS. 8A-8B and 9A-9B illustrate operation of the handle assembly 40 in an initial configuration providing an open/close functionality to the jaw assembly 30. In FIG. 8A, the movable trigger 44 is at an open position, and the actuation shaft 60 is at a first position, corresponding to the first position of the actuation beam at the distal end of the elongate shaft 20. In the initial position, the actuation shaft 60 is positioned at the second orientation such that the reverse driver 54 is angularly aligned with the reversing surface 64. With actuation shaft 60 in the second orientation, the opening driver 58 is positioned within the opening surface 66 or recess. Movement of the movable handle 44 from the open position (FIG. 8A-8B) to the closed position (FIG. 9A-9B), advances the forward driver 52 distally along the actuation shaft 60 to engage an advancing recess 63 formed in the actuation shaft 60 and drive the actuation shaft 60 distally in the handle assembly 40 to a second position. The second position of the actuation shaft 60 within the handle assembly 40 corresponds to the second position of the actuation beam, which positions the jaw assembly 30 in a closed configuration.

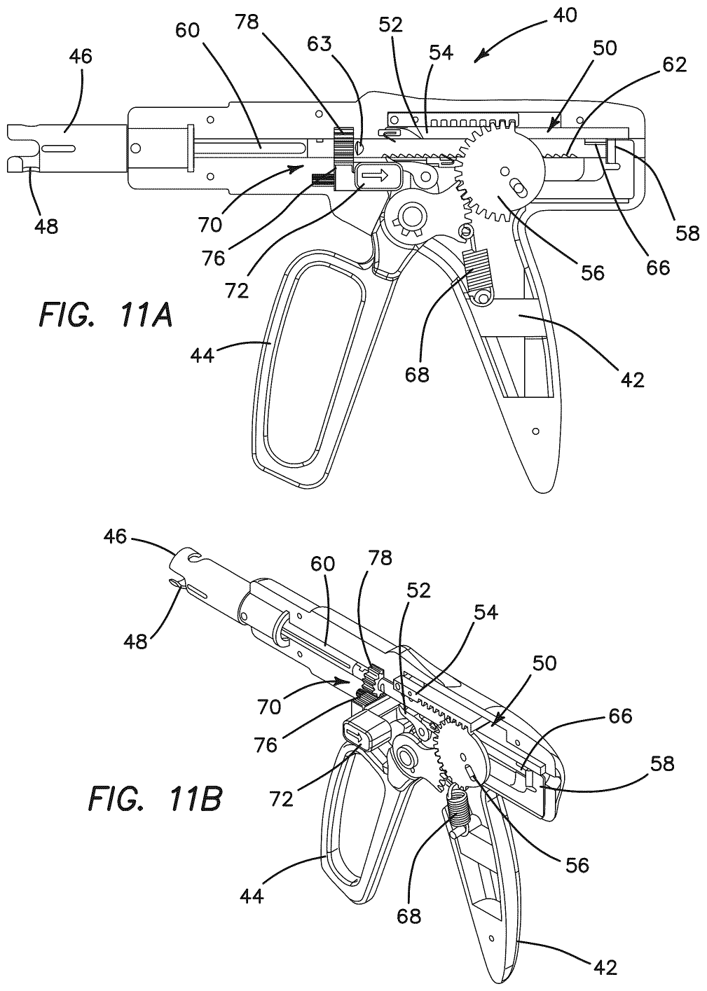

The movable handle 44 can be biased to the open position by a biasing member, such as a coil spring 68 (FIG. 11A). Thus, releasing the movable handle 44 from the closed position illustrated in FIG. 9A-9B would return it to the open position of FIGS. 8A-8B. Operable coupling of the movable handle 44 to the opening driver 58 would likewise translate the opening driver 58 proximally within the handle assembly 40 as the movable handle 44 returns to the open position. In the second orientation of the actuation shaft 60, the opening driver 58 engages opening surface 66 such that the proximal movement of the opening driver 58 returns the actuation shaft 60 from the second position to the first position, returning the jaw assembly 30 to the open configuration.

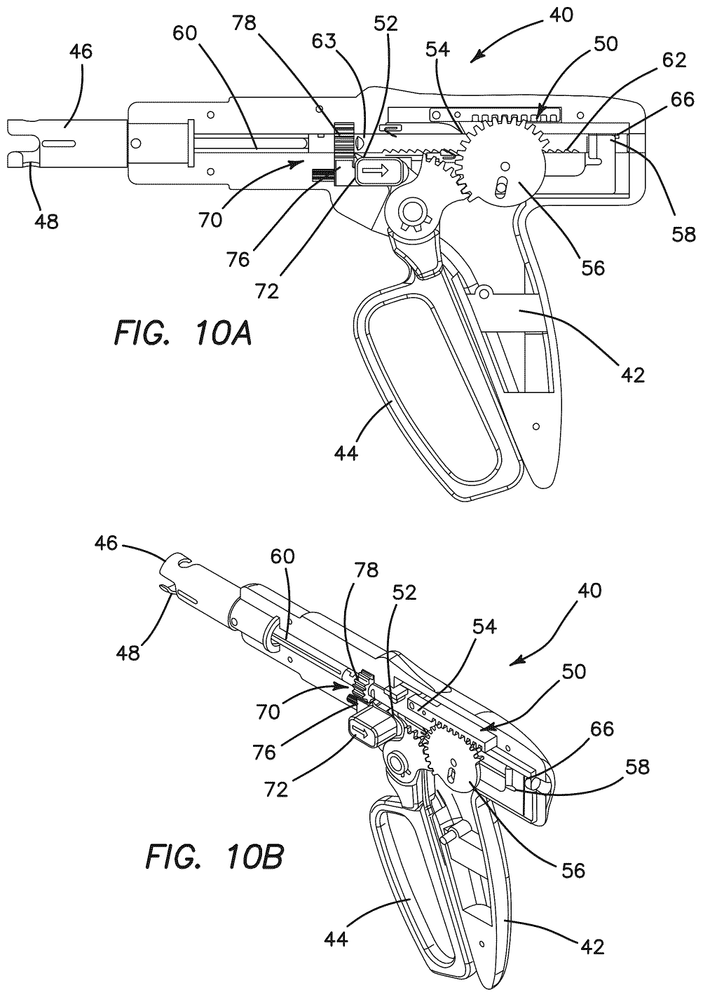

A user can seek a desired stapling position within a surgical field by repeatedly opening and closing the jaws to clamp tissue in various locations. Once a desired stapling position has been selected, the actuation mechanism 50 can be configured in a stapling or firing mode by rotating the actuation shaft 60 to the first orientation. With the jaw assembly a closed configuration at a desired stapling position (as illustrated in FIGS. 9A-9B), a user can reposition the selector 72 by sliding the slider to the first position, corresponding to the first orientation of the actuation shaft 60 (as illustrated in FIGS. 10A-10B). In the first orientation of the actuation shaft 60, the forward driver 52 is engageable with the advancing surface 62, the reversing driver 54 is angularly misaligned with the reversing surface 64, and the opening driver 58 angularly misaligned with the opening surface 66. With the actuation shaft 60 in the first orientation, the movable handle 44 can be released into the open position (FIG. 11A-11B), engaging the forward driver 52 with the advancing surface 62.

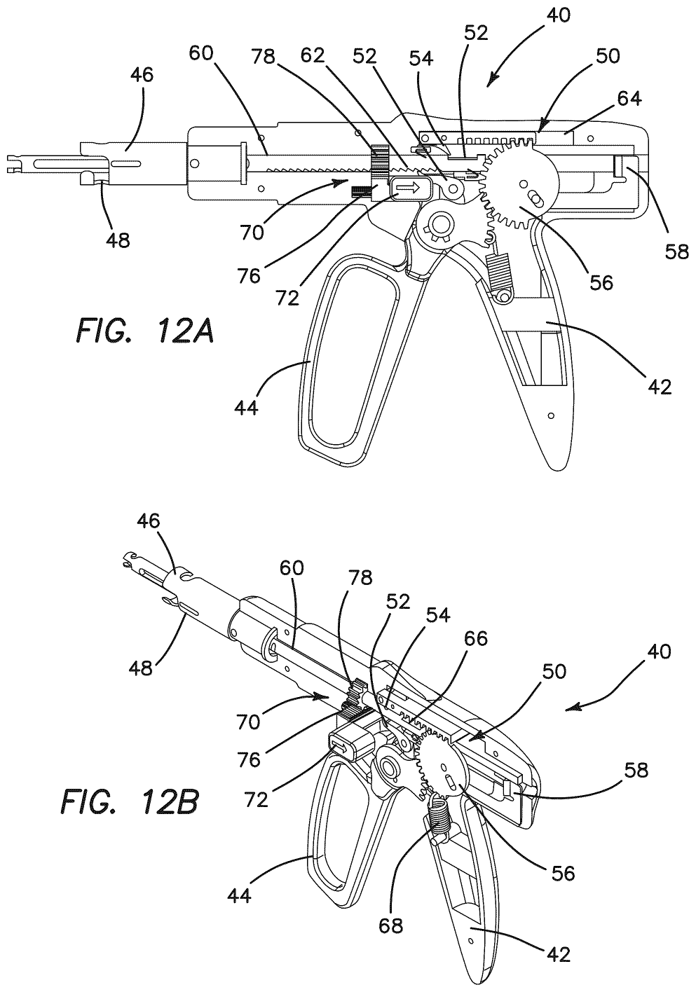

With reference to FIGS. 11A-11B and 12A-12B, with the actuation shaft 60 in the first orientation, and the forward driver 52 engaging the advancing surface 62, the actuation mechanism 50 is in a stapling or firing mode. Several cycles of movable handle 44 movement from the open position to the closed position and back to the open position advance the actuation shaft 60 from the second position (FIGS. 11A-11B), to a third position in which the actuation shaft 60 is moved to its distal-most limit with respect to the handle assembly 40 (FIGS. 12A-12B). In some embodiments, the actuation mechanism can include a stop to interfere with distal travel of the actuation shaft 60 at the third position. The second position of the actuation shaft corresponds to the second position of the actuation beam in the jaw assembly 30. The third position of the actuation shaft corresponds to the third position of the actuation beam in the jaw assembly 30 in which the plurality of staples have been deployed from the first jaw. With movement of the movable handle 44 or trigger in the firing mode to advance the actuation shaft from the second position to the third position, the forward driver 52 is sequentially advanced over the adjacent teeth or grooves of the actuating surface 62 in a ratchet-like advancement.

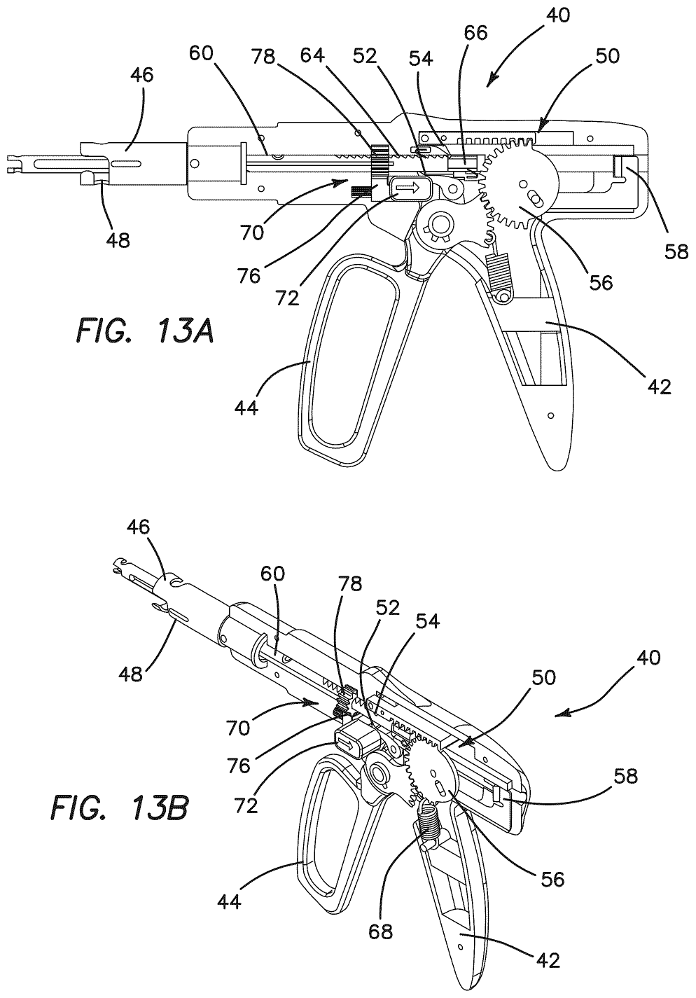

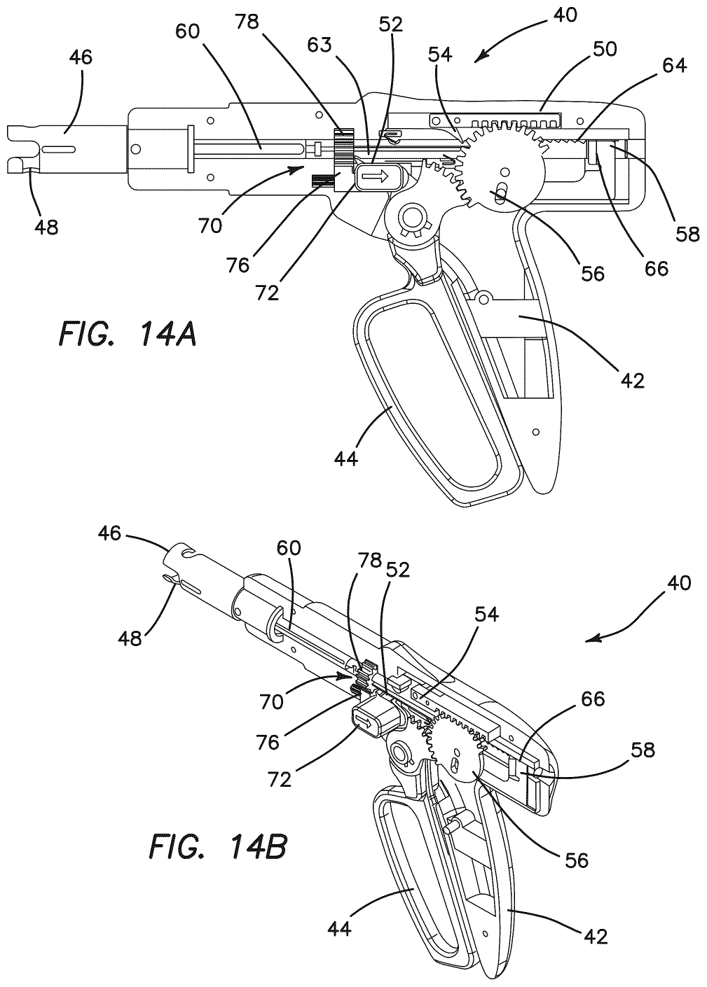

With reference to FIGS. 13A-13B, once the actuation shaft 60 has been advanced to the third position and the staples have been fired from the jaw assembly, the actuation mechanism 50 can be configured in a reverse mode. Accordingly, the rotation mechanism 70 can rotate the actuation shaft 60 to the second orientation to position the reversing surface 64 in angular alignment with the reverse driver 54. The slider can be slid to the second position to rotate the actuation shaft from the first orientation (FIGS. 12A-12B) to the second orientation (FIGS. 13A-13B). With the actuation shaft 60 in the second orientation, repeated cycles of the movable handle 44 from the open position to the closed position and back to the open position engage the reverse driver 54 with the reversing surface 64 in a ratchet-like advancement while retracting the actuation shaft 60 proximally in the handle assembly 40. Once the reverse driver 54 has driven the actuation shaft 60 proximally to the second position (illustrated in FIGS. 14A-14B), the opening driver 58 engages the opening surface 66. The opening driver 58 returns the actuation shaft 60 to the first position when the movable handle 44 is released to the open position. (Returning the handle assembly to the configuration illustrated in FIGS. 8A-8B). With the actuation shaft 60 in the first position, the cartridge, emptied of staples, can be decoupled from the handle assembly 40 and a new cartridge can be coupled to the handle assembly to begin another stapling operation.

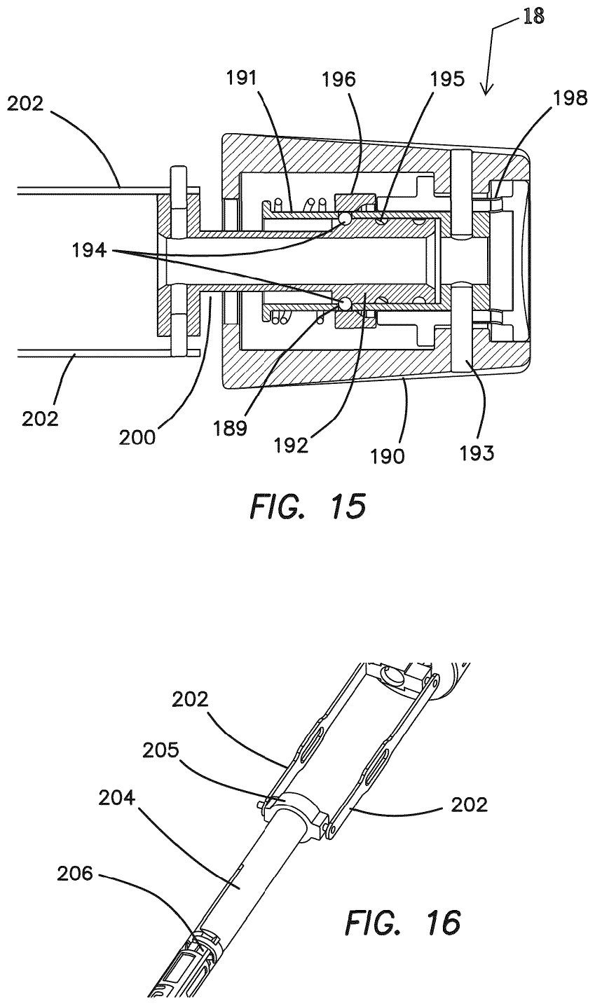

With reference to FIGS. 15, 16, and 17A-17F, an embodiment of articulation mechanism 18 for the handle assembly 40 is illustrated. In the illustrated embodiment, the handle can articulate the jaw assembly at the distal end of the shaft up to 45° in a fully articulated position in either direction relative to a longitudinally centered position. In some embodiments, the handle assembly uses a manual articulation mechanism 18 including a series of components coupled to the manually actuated articulation knob 190 at the proximal end of the handle. It is contemplated that in other embodiments, the articulation knob and certain components of the articulation mechanism can be disposed at other locations on the handle assembly such as, for example, the distal end, an upper surface thereof, or on the stationary handle.

With reference to FIGS. 15 and 16, the articulation mechanism is coupled to an articulation member 206 extending longitudinally within the reload shaft when the reload shaft is coupled to the handle. Actuation of the articulation mechanism longitudinally translates the articulation member 206 proximally or distally relative to the shaft to articulate the jaw assembly at the distal end of the shaft.

With reference to FIG. 15, the articulation mechanism comprises a ball screw 192 having at least one helical groove or thread 195 in which one or more ball bearing 194 can ride. In the illustrated embodiment, the articulation mechanism comprises two ball bearings 194 that are engageable in two threads 195. The ball bearings 194 are positioned in ball bearing apertures 189 in a ball sleeve 191 positioned radially outwardly of the ball screw 192. The ball bearings 194 are maintained in the threads 195 by a release sleeve 196 positioned radially outward of the ball bearings 194. Rotation of the articulation knob 190, which is coupled to the ball sleeve 191 such as by connecting pins 193, rotates the ball sleeve 191 about an axis of rotation, causing the ball bearings 194 to travel within the threads 195 and correspondingly longitudinally translate the ball screw 192. Articulation of the jaw assembly is accomplished by rotating the articulation knob 190 to correspondingly rotate the ball sleeve 191 and the ball bearings 194 about the axis of rotation while their longitudinal position is fixed along the axis of rotation. The ball bearings 194, which are engaged in the threads 195 of the ball screw 192 will then translate the ball screw 192 forward and reverse along the axis of rotation. In the illustrated embodiment, the ball sleeve 191 is generally tubular, having a cavity formed therein, and a portion of the ball screw 192 is positioned within the cavity and translates longitudinally within the cavity. While the illustrated embodiment of articulation mechanism includes two ball bearings engageable threads in a ball screw, it is contemplated that in other embodiments, the articulation mechanism can have fewer or more than two ball bearings such as, for example, a single ball bearing positioned in a single helical screw or three or more ball bearings in a corresponding number of helical threads.

With reference to FIGS. 15 and 16, the ball screw 192 extends to a distal end 200 coupled to a pair of articulation links 202. The articulation links 202 are spaced apart from one another, which desirably allows them to be positioned radially outwardly of the actuation mechanism and actuation shaft within the handle. As illustrated in FIG. 16, the articulation links 202 can comprise a mating feature such as a slot formed therein to allow them to be keyed into a corresponding mating feature such as a post extending radially inwardly from the handle body. The slots can stabilize the articulation links relative to the handle and interaction of the handle posts with ends of the slots can define a range of articulation for the articulation mechanism. The distal ends of the articulation links 202 can be rotatably coupled to the articulation adapter 204, which can be positioned coaxially radially outwardly of the actuation adapter at the distal end of the handle. This rotational coupling can include an articulation bearing 205 having relatively low friction properties. This articulation bearing 205 can facilitate rotation of a coupled reload shaft relative to the handle assembly and longitudinal movement of the articulation adapter 204 during operation of the articulation mechanism. While the illustrated embodiment of articulation mechanism includes two articulation links laterally offset from the actuation mechanism within the handle, it is contemplated that in other embodiments, the articulation mechanism can have fewer or more than two articulation links such as, for example, an articulation link or three or more articulation links.

With continued reference to FIG. 16, the articulation adapter 204 can be connected to the articulation member 206 in the shaft by a bayonet connection when the shaft is coupled to the handle. The articulation member 206 extends distally within the shaft and is coupled to an end effector or jaw assembly articulably coupled to the shaft. The threads 195 can be configured such that moving the ball screw proximally will articulate the jaw assembly to the left when viewed from the handle relative to a longitudinally centered position and moving the ball screw 192 distally will articulate the jaw assembly to the right when viewed from the handle relative to the centered position.

Advantageously, since the helical threads 195 of the ball screw 192 are continuous, the articulation mechanism can allow the jaw assembly to be articulated to virtually infinite angular positions between a desired operational range. In some embodiments, the articulation mechanism can be configured to provide an articulation operational range from −45° to +45° of the jaw assembly relative to a longitudinally centered position defined by the longitudinal axis of the shaft. In other embodiments, the articulation mechanism can be configured to provide other operative articulation ranges including ranges providing more than +/−45° of articulation or those providing less than +/−45° of articulation. In some embodiments, the articulation mechanism can be configured to provide articulation in a single direction relative to a longitudinally centered position.

In some embodiments, the pitch of the threads 195 on the ball screw 192 is variable. For example, the threads 195 can include a relatively low pitch towards an end of the threads to advantageously provide a larger mechanical advantage when the jaw assembly can require more force to articulate. The threads 195 can include a relatively higher pitch towards a center of the threads to allow rapid movement with a relatively lower mechanical advantage where the jaw assembly can require a lower force to articulate. In other embodiments, the threads 195 include a constant pitch such that rotation of the articulation knob results in a proportional amount of articulation of a jaw assembly of the stapler that does not vary over the articulation range of the articulation mechanism. Desirably, such a constant pitch thread ball screw can result in an easily predictable response during operation of the actuation mechanism.

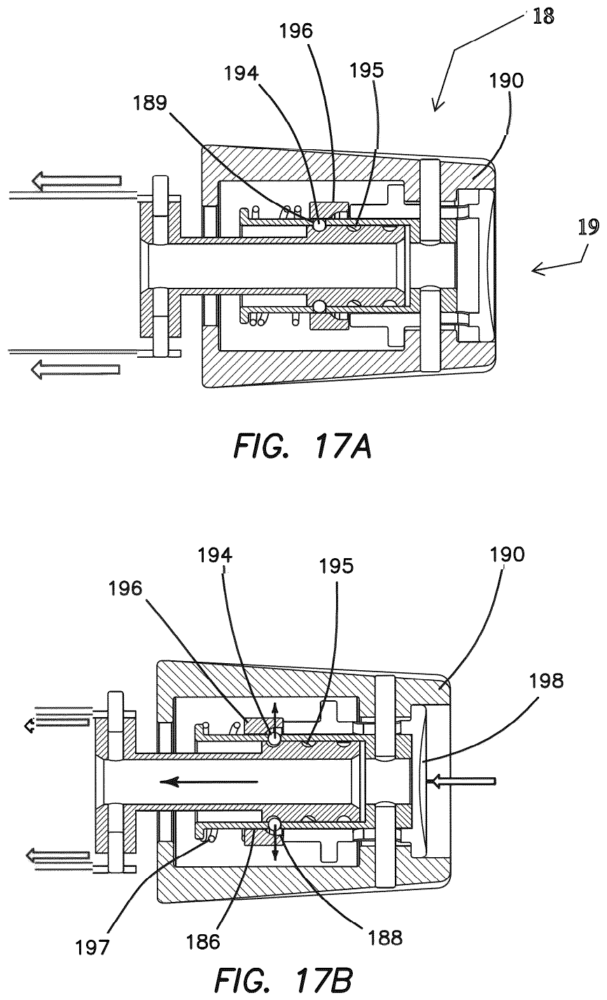

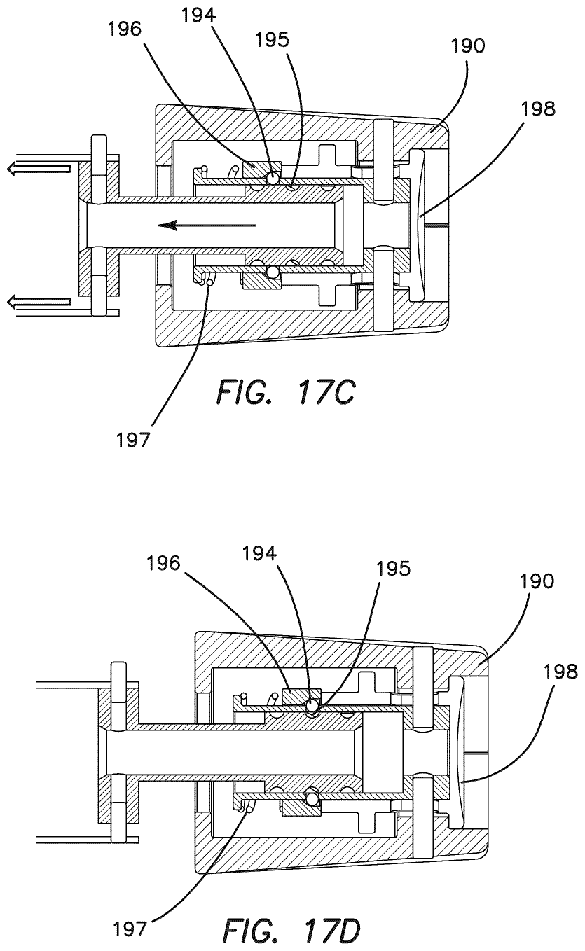

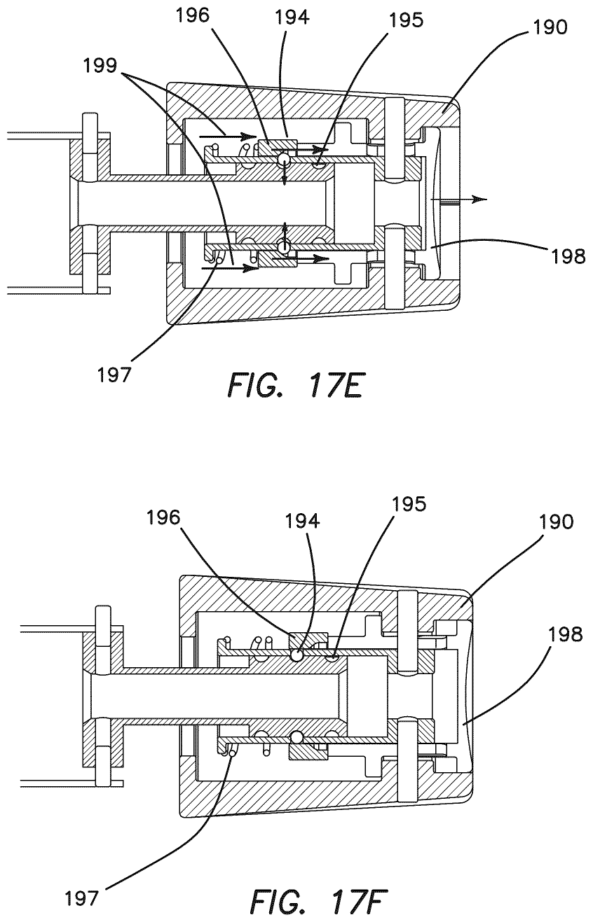

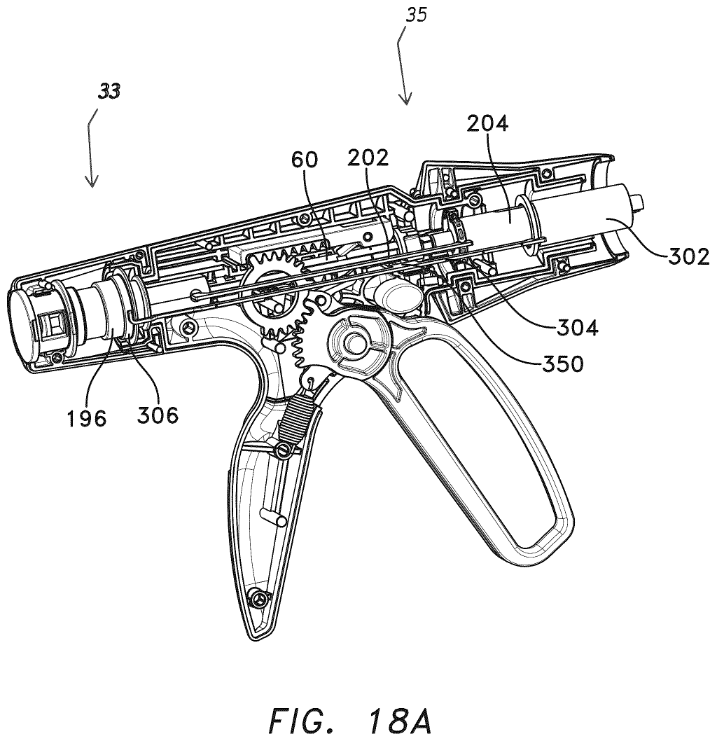

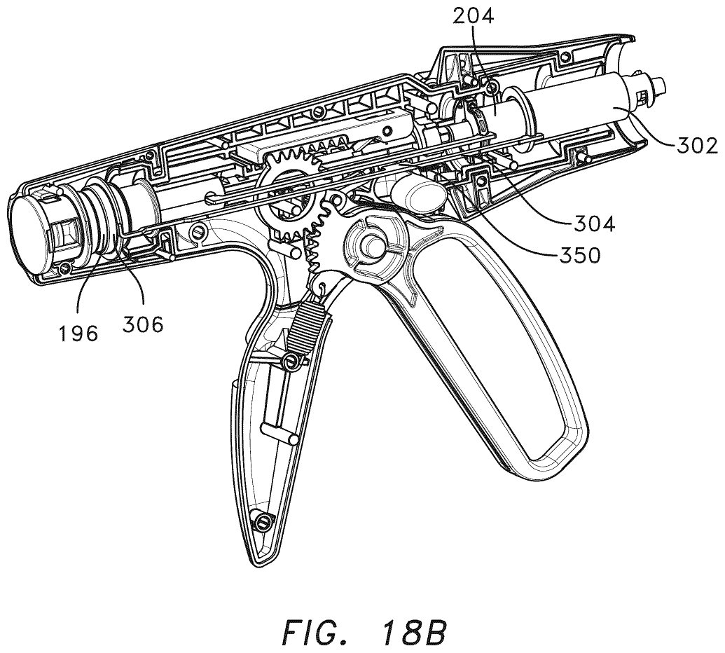

With reference to FIGS. 17A-17F, the articulation mechanism 18 can comprise a release mechanism 19 that allows the articulation mechanism 18 to advantageously be reset to the longitudinally centered position from any articulated position. The release mechanism 19 is operated by user pressing a release button 198. In the illustrated embodiment, the release button 198 is positioned radially nested within the articulation knob 190.

With reference to FIG. 17B, operation of the release button 198 will distally advance the release sleeve 196. A radially inner surface of the release sleeve 196 is stepped to include an engagement surface 186 having a relatively small inner diameter and a release surface 188 having a relatively larger inner diameter with a smooth ramp between the engagement surface and the release surface. In operation, the engagement surface of the release sleeve maintains the ball bearings 194 in the threads 195 of the ball screw 192. Once the release button 198 is pushed, the engagement surface is distally advanced, allowing the ball bearings 194 to disengage from the threads 195 and advance radially outward through the ball bearing apertures 189 in the ball sleeve 191 (FIG. 15) against the release surface.

With reference to FIGS. 17C and 17D, with the ball bearings 194 disengaged from the threads 195, the articulation mechanism can be biased to a centered position. In some embodiments, the ball screw 192 is biased to a centered position by a biasing member such as two springs 197 and spring force from the shaft. The ball bearings 194 positioned in the centered position (FIG. 17D) along the threads 195 corresponds to a longitudinally centered position of the jaw assembly.

With reference to FIGS. 17E-17F, once the release button 198 is allowed to return to an undisturbed configuration, release sleeve 196 is retracted proximally (indicated by arrows 199) by a spring. Proximal movement of the release spring 196 forces the ball bearings 194 into engagement with the threads 195 of the ball screw. Thus, the articulation mechanism can then be used to articulate the jaw assembly from the longitudinally centered position, or the stapler can be used with the jaw assembly in the longitudinally centered position.

In certain embodiments, the handle assemblies described herein can further comprise an articulation lockout mechanism and a shaft coupling firing lockout mechanism. The articulation lockout mechanism can be configured to prevent operation of the articulation mechanism with no reload shaft coupled to the handle assembly and allow operation of the articulation mechanism as described above with respect to FIGS. 15-17A when a reload shaft is coupled to the handle assembly. Desirably, this articulation lockout mechanism facilitates coupling the articulation mechanism as described with respect to FIG. 16 as the reload shaft is coupled to the handle assembly. If the articulation mechanism were maintained in an engaged configuration even when no instrument shaft were coupled to the handle assembly, it could be difficult to align the articulation member within the instrument shaft with the articulation adapter 204 in an attempt to couple the instrument shaft with the handle assembly.

The shaft coupling firing lockout mechanism can be configured to prevent a user from selecting a firing mode of the handle assembly unless a reload shaft is coupled to the handle assembly. Thus, desirably, a user is prevented from initiating a firing operation if the reload shaft is not secured to the handle assembly. In the illustrated embodiment, the articulation lockout mechanism and shaft firing lockout mechanism are integrated and share certain components. This integrated mechanism can desirably present manufacturing and packaging efficiencies. It is contemplated that in other embodiments a handle assembly can include an articulation lockout mechanism that is distinct from a shaft coupling firing lockout mechanism. Moreover, in other embodiments, a handle assembly can include only one of an articulation lockout mechanism and a shaft coupling firing lockout mechanism.

With reference to FIGS. 18A-18B, a perspective view of an embodiment of handle assembly having an articulation lockout mechanism 33 and a shaft coupling firing lockout mechanism 35 are illustrated with a portion of a housing of the handle assembly removed to illustrate the mechanisms therein. As illustrated in FIG. 18A, the articulation lockout mechanism 33 and shaft coupling firing lockout mechanism 35 are in a locked out configuration corresponding to no reload shaft coupled to the handle assembly. As illustrated in FIG. 18B, the articulation lockout mechanism and shaft coupling firing lockout mechanism are in an unlocked configuration corresponding to a reload shaft coupled to the handle assembly.

With continued reference to FIGS. 18A-18B, the articulation lockout mechanism comprises a lockout sleeve 302 at the distal end of the handle assembly and at least one lockout arm 304 coupled to the lockout sleeve. In the illustrated embodiment, the lockout sleeve 302 can be positioned radially outwardly of the articulation adapter 204. In the illustrated embodiment, the articulation lockout mechanism comprises two lockout arms 304 extending longitudinally within the handle assembly from a proximal end coupled to a locking sleeve 306 positioned around the release sleeve 196 of the articulation mechanism to a distal end coupled to the lockout sleeve 302. The lockout arms 304 can extend parallel to and offset from the articulation links 202 of the articulation mechanism. The lockout arms can be positioned laterally outwardly of the actuation shaft 60 and other actuation mechanism components. In other embodiments, one or more than two lockout arms 304 can couple the lockout sleeve 302 to the release sleeve 196, and the lockout arms 304 can be disposed in a different lateral position than in the illustrated embodiment.

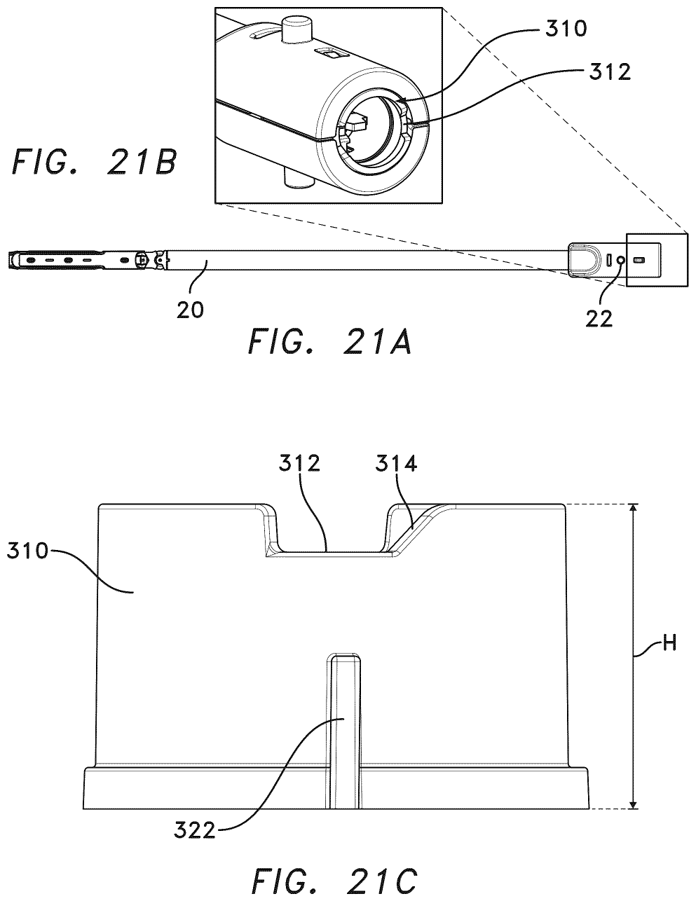

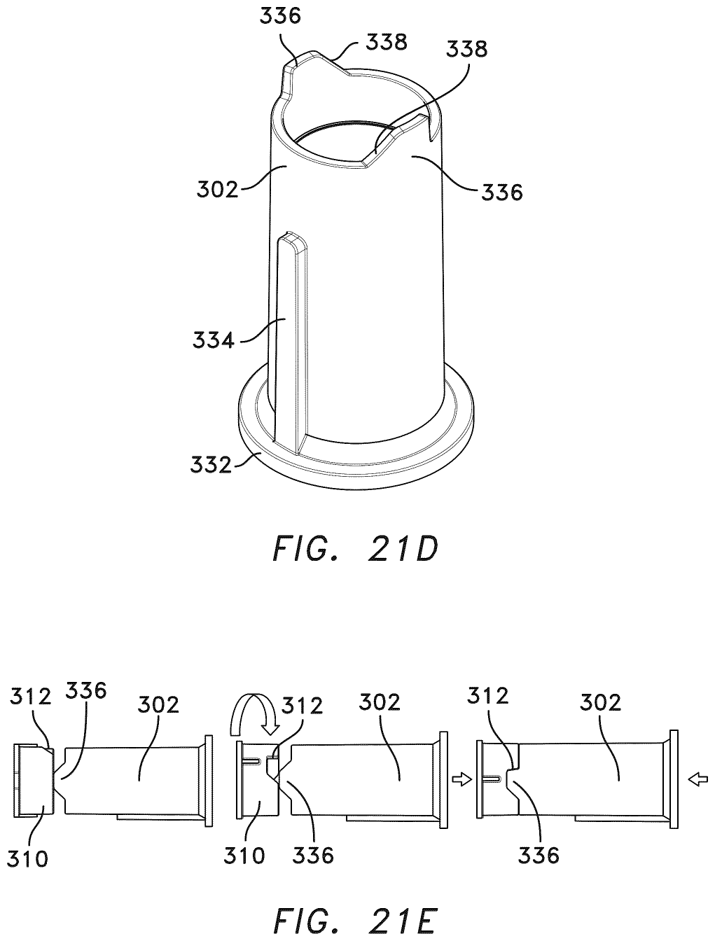

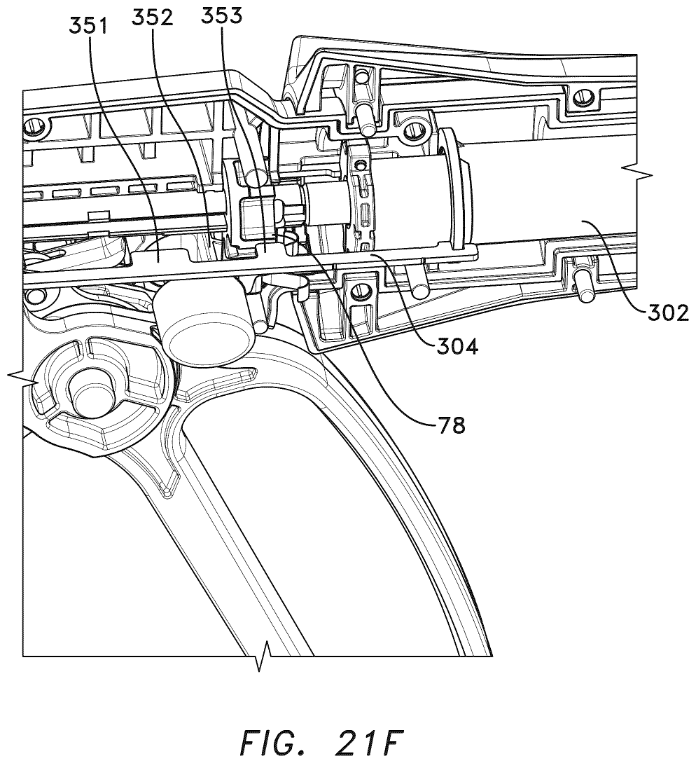

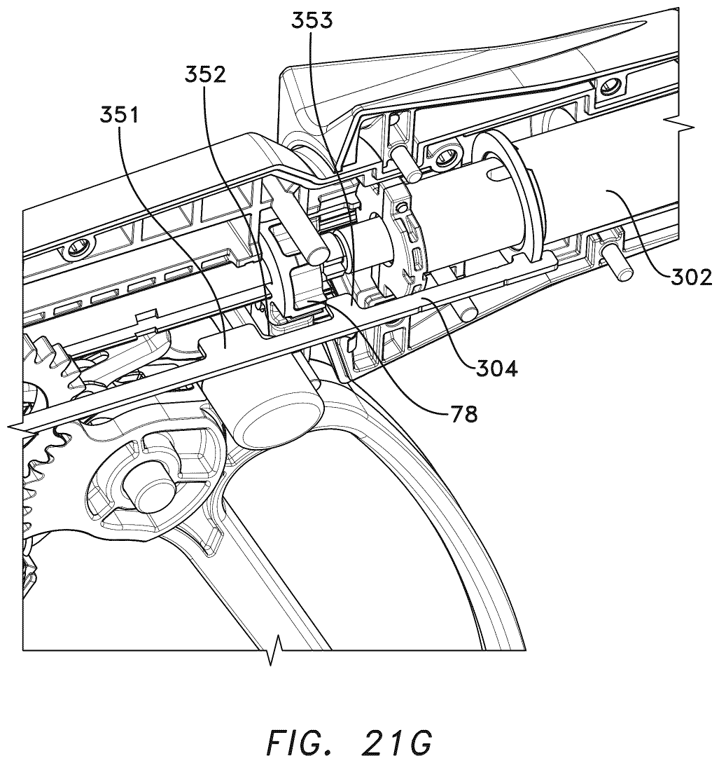

With continued reference to FIGS. 18A-18B, in the illustrated embodiment, the shaft coupling firing mechanism comprises the lockout sleeve 302 at the distal end of the handle assembly and the at least one lockout arm 304 coupled to the lockout sleeve 302 and extending proximally therefrom. The at least one lockout arm 304 comprises at least one lockout tab 350 extending radially inwardly therefrom. In the illustrated embodiment, the articulation lockout mechanism comprises two lockout arms 304, on laterally opposed sides of the actuation shaft 60, and one of the two lockout arms 304 comprises one lockout tab 350. As further discussed with reference to FIGS. 21F and 21G, in certain embodiments to provide lock out functionality during engagement of an instrument shaft with the handle assembly, a lockout arm 304 can comprise two lockout tabs: a proximal lockout tab 351 and a distal lockout tab 353 with a lockout recess 352 therebetween.

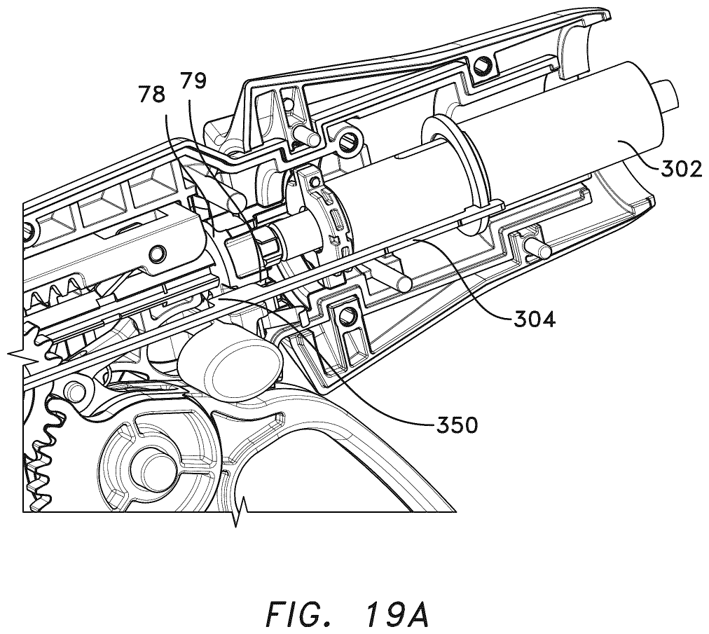

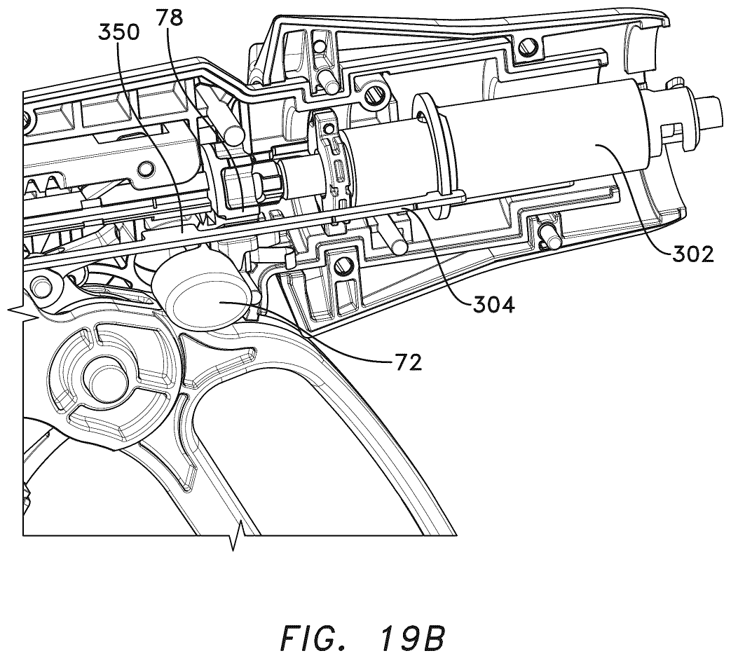

With reference to FIGS. 19A-19B, perspective views of the shaft coupling firing lockout mechanism are illustrated. As illustrated in FIG. 19A, the shaft coupling firing lockout mechanism is in a locked out configuration corresponding to no reload shaft being coupled to the handle assembly. As illustrated in FIG. 19B, the shaft coupling firing lockout mechanism is in an unlocked configuration corresponding to a reload shaft being coupled to the handle assembly.

With reference to FIG. 19A, the shaft coupling firing lockout mechanism is biased longitudinally distally. For example, in some embodiments a coil spring can bias the lockout sleeve 302 distally relative to the handle assembly. With no reload shaft coupled to the handle assembly, the lockout sleeve 302 and lockout arm 304 are biased to a distal position corresponding to the locked out configuration of the shaft coupling firing lockout configuration. With the lockout arm 304 in the distal position, the lockout tab 350 is longitudinally aligned with the gear 78 of the rotation mechanism. In this position, the lockout tab 350 prevents rotation of the gear 78 such that a user is unable to rotate the actuation shaft 60 to a firing orientation. In certain embodiments, the gear 78 can comprise a slot 79 positioned to receive the lockout tab of the lockout arm 304

With reference to FIG. 19B, as a reload shaft is coupled to the handle assembly, a lockout keyway 312 (FIG. 21A-21B) engages the lockout sleeve 302 and advances the lockout sleeve 302 and lockout arm 304 longitudinally proximally a predetermined distance to a proximal position. In the proximal position, as illustrated, the lockout tab 350 is misaligned with the gear 78 of the rotation mechanism such that the rotation mechanism can be actuated by the selector 72 as described with respect to FIGS. 8-14.

Thus, desirably, the shaft coupling firing lockout mechanism prevents initiating a firing actuation of the handle assembly without a reload shaft fully coupled to the handle assembly. Desirably, this lockout mechanism can facilitate proper alignment of the bayonet coupling features of the handle assembly and reload shaft during an initial coupling of the reload shaft to the handle assembly. Furthermore, the lockout mechanism can desirably prevent an inadvertent attempt to fire a reload shaft that is not securely, fully coupled to the handle assembly.

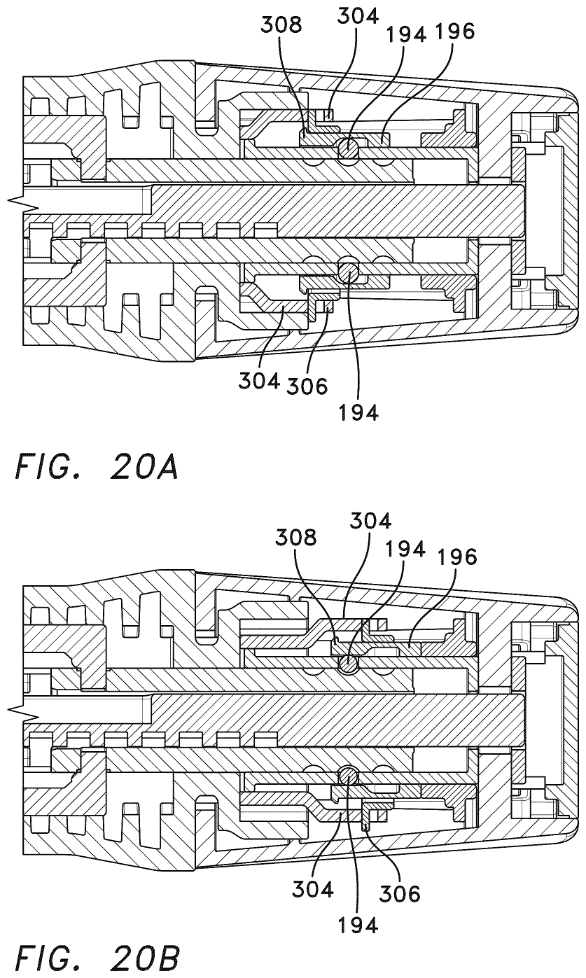

With reference to FIGS. 20A-20B, cut away top views of the articulation lockout mechanism are illustrated. As illustrated in FIG. 20A, the articulation lockout mechanism is in a locked out configuration corresponding to no reload shaft being coupled to the handle assembly. As illustrated in FIG. 20B, the articulation lockout is in an unlocked configuration corresponding to a reload shaft being coupled to the handle assembly.

With reference to FIG. 20A, with no reload shaft coupled to the handle assembly, the lockout sleeve at the distal end of the handle assembly is biased to the distal position, advancing the lockout arms 304 and locking sleeve 306 to a distal position. As illustrated, the locking sleeve 306 is positioned around the release sleeve of the articulation mechanism. In embodiments of handle assembly having an articulation lockout, the release sleeve 196 can comprise a flange 308 protruding radially outwardly at a distal end of the release sleeve. As the locking sleeve 306 is biased to the distal position corresponding to a locked out configuration of the articulation lockout mechanism, the locking sleeve 306 engages the flange 308 of the release sleeve 196, moving the release sleeve distally to release ball bearings 194 from the ball screw as described above with reference to FIGS. 17B-17D. Accordingly, with no reload shaft coupled to the handle assembly, the articulation knob may be rotated without actuating the articulation mechanism because the ball bearings 194 are disengaged from the threads of the ball screw.

Thus, in certain embodiments, the articulation lockout mechanism can maintain the articulation mechanism in a centered position if no instrument shaft is coupled to the handle assembly. This centered position of the articulation adapter can facilitate the bayonet coupling of instrument shaft and handle assembly previously discussed above. If the articulation mechanism were maintained in an engaged configuration even when no instrument shaft were coupled to the handle assembly, it could be difficult to align the articulation member within the instrument shaft with the articulation adapter 204 in an attempt to couple the instrument shaft with the handle assembly.

With reference to FIG. 20B, once a reload shaft has been coupled to the handle assembly, the lockout sleeve at the distal end of the handle assembly is advanced to the proximal position, advancing the lockout arms 304 and locking sleeve 306 to a proximal position. As illustrated, with the locking sleeve 306 in the proximal position, the locking sleeve 306 is spaced apart from the flange 308 of the release sleeve. 196 Accordingly, with the articulation lockout mechanism in the unlocked configuration, ball bearings 194 are engaged with the ball screw, as described above with respect to FIG. 17F, and the articulation mechanism and its release button are operable as described above with respect to FIGS. 15-17F. Thus, with an instrument shaft attached, rotation of the articulation knob results in translation of the articulation adapter to articulate an end effector coupled to the instrument shaft.

While in certain embodiments, the shaft coupling firing lockout mechanism can have a locked out configuration in which the lockout sleeve is biased to the distal position and an unlocked configuration in which a shaft has been coupled to the handle assembly, as discussed above with respect to FIGS. 19A-19B, in certain embodiments, the reload shaft and shaft coupling firing lockout mechanism can be configured to further comprise an engagement position of the lockout mechanism in which the reload shaft is being coupled to the handle assembly but not yet fully seated. It can be desirable that the reload shaft is securely coupled to the handle assembly before a firing operation is initiated to facilitate the reliable firing of staples. Thus, in certain embodiments, the shaft coupling firing lockout mechanism can be configured to position the lockout sleeve and lockout arm in a locked out configuration while the reload shaft is being coupled to the handle assembly. In certain embodiments, a lockout keyway on the reload shaft can be sized and configured to engage the lockout sleeve of the handle assembly to initially position the lockout sleeve and the shaft coupling firing lockout mechanism in the engagement position during coupling of the shaft with the handle assembly, then position the lockout sleeve and the shaft coupling firing lockout mechanism in the proximal position once the reload shaft is securely coupled to the handle assembly.

With reference to FIGS. 21A-21C, an embodiment of reload shaft for use with a handle assembly having a shaft coupling firing lockout mechanism and an articulation lockout mechanism is illustrated. FIG. 21A illustrates a side view of the reload shaft 20 having a proximal end 22 configured to couple to the coupler 46 of the handle assembly. (FIGS. 1-2). FIG. 21B is a detail perspective view of the proximal end 22 of the reload shaft 20 having a lockout keyway 310 therein. The lockout keyway 310 comprises at least one notch 312 formed therein that is engageable with the lockout sleeve to sequentially position the shaft coupling firing lockout mechanism initially in the engagement position corresponding to a locked out configuration of the mechanism, then to the proximal position, corresponding to an unlocked configuration of the mechanism.

With reference to FIG. 21C, an exemplary embodiment of lockout keyway 310 is illustrated. The illustrated lockout keyway 310 has a height H to a proximal edge, a notch 312 recessed from the height H. The lockout keyway comprises a ramped edge 314 extending between the proximal edge and at least one side of the notch 312. Each of the lockout keyways further comprise a key, such as a rib 322 to restrict rotation of the lockout keyway relative to the reload shaft as the reload shaft is coupled with the handle assembly.

With reference to FIG. 21D, an exemplary lockout sleeve 302 of the handle assembly is illustrated. In the illustrated embodiment, the lockout sleeve 302 comprises an engagement feature such as a flange 332 at a proximal end and at least one rib 334 or other key element protruding from the outer surface thereof to maintain an orientation of the lockout sleeve 302 relative to a longitudinal axis of the actuation shaft. As illustrated, the lockout sleeve 302 further comprises at least one mating protrusion such as a tooth 336 extending distally from the distal end thereof positioned to engage a corresponding notch of a lockout keyway of a connected reload shaft. The at least one tooth 336 can have a ramped edge 338 such that it can matingly engage a notch of a lockout keyway that likewise has a ramped edge. In the illustrated embodiment, the lockout sleeve 302 comprises two teeth 336 positioned diametrically opposed on a distal end of the lockout sleeve 302 to engage a corresponding two identification notches. In other embodiments, it is contemplated that the number and locations of mating features included the lockout keyways and lockout sleeves can be varied.

With reference to FIG. 21E, an exemplary sequence of interaction between a lockout sleeve 302 and a lockout keyway 310 is illustrated as a reload shaft is installed on a handle assembly. As illustrated, the shaft installation sequence proceeds from left to right. In the left panel, as the shaft is positioned in the coupler 46 (FIG. 1) of the handle assembly, the lockout sleeve 302 is oriented such that the teeth 336 are misaligned with the notches 312. The coupler 46 and shaft engage in a bayonet connection in which the shaft is advanced longitudinally proximally relative to the handle, then rotated relative to the longitudinal axis. The center panel illustrates the proximal longitudinal movement longitudinally proximally displacing the lockout sleeve 302 relative to the handle as the rotational movement of the shaft moves the teeth 336 closer to alignment with the notches 312. The right panel illustrates completion of rotation of the shaft relative to the handle assembly to secure the bayonet coupling. As illustrated, once the shaft is coupled to the handle assembly, the teeth 336 of the lockout sleeve 302 engage and are positioned within the notches 312 of the lockout keyway 310. Thus, during a coupling operation, the lockout sleeve 302 is initially displaced proximally by installation of the shaft with the teeth misaligned with the notches, then returns distally as the teeth 336 engage the notches 312. Thus, in the illustrated embodiment as a bayonet coupling is initiated, the lockout sleeve is advanced longitudinally proximally to the engagement position of the shaft coupling firing lockout mechanism. Once the reload shaft has been rotated with respect to the handle assembly to complete the bayonet coupling, the lockout sleeve is biased distally to the proximal position corresponding to an unlocked configuration of the shaft coupling firing lockout mechanism.

With reference to FIG. 21F, a side view of the shaft coupling firing lockout mechanism is illustrated in the engagement position. In the illustrated embodiment, the lockout arm 304 comprises two lockout tabs 351, 353 separated by a lockout recess 352 to provide a locked out configuration with the lockout arm 304 in the engagement position. Initially with no reload shaft coupled to the handle assembly, the shaft coupling firing lockout mechanism is positioned in a locked out configuration similar to FIG. 19A. In this locked out configuration, the lockout sleeve 302 and lockout arm 304 are biased to a distal position such that the proximal lockout tab 351 is positioned to prevent rotation of the gear 78 of the rotation mechanism. As the reload shaft is advanced proximally in a bayonet coupling operation with the handle assembly, the lockout sleeve 302 and lockout arm 304 are advanced proximally to the engagement position of the shaft coupling firing lockout mechanism in which the distal lockout tab 353 is positioned to prevent rotation of the gear 78 of the rotation mechanism.

With reference to FIG. 21G, a side view of the shaft coupling firing lockout mechanism of FIG. 21F is illustrated in the proximal position corresponding to an unlocked configuration of the lockout mechanism. Once the reload shaft is rotated to securely couple the reload shaft to the handle assembly, the lockout sleeve 302 and lockout arm 304 are biased distally to the proximal position of the shaft coupling firing lockout mechanism in which the lockout recess 352 is positioned to allow rotation of the gear 78 of the rotation mechanism. Thus, with the lockout arm 304 in the proximal position as illustrated, the shaft coupling firing lockout mechanism is in the unlocked configuration.

Although this application discloses certain preferred embodiments and examples, it will be understood by those skilled in the art that the present inventions extend beyond the specifically disclosed embodiments to other alternative embodiments and/or uses of the invention and obvious modifications and equivalents thereof. Further, the various features of these inventions can be used alone, or in combination with other features of these inventions other than as expressly described above. Thus, it is intended that the scope of the present inventions herein disclosed should not be limited by the particular disclosed embodiments described above, but should be determined only by a fair reading of the claims.

What is claimed is:

1. A handle assembly for a surgical stapler, the handle assembly comprising:
a handle body comprising a stationary handle and a movable handle pivotably coupled to the handle body, the handle body comprising a coupler configured to removably couple to an instrument shaft having a stapler jaw assembly;
an actuation shaft mechanically coupled to the movable handle for manual actuation thereof, the actuation shaft slidable within the handle body along a longitudinal axis, the actuation shaft selectively rotatable between a firing orientation in which movement of the movable handle longitudinally slides the actuation shaft a first direction and a reverse orientation in which movement of the movable handle longitudinally slides the actuation shaft a second direction opposite the first direction;
an articulation mechanism configured to articulate the stapler jaw assembly relative to the instrument shaft when the instrument shaft is coupled to the handle body:
an articulation lockout mechanism preventing operation of the articulation mechanism when no instrument shaft is coupled to the coupler and allowing operation of the articulation mechanism when the instrument shaft is coupled to the coupler; and
a shaft coupling firing lockout mechanism, the shaft coupling firing lockout mechanism preventing rotation of the actuation shaft to the firing orientation when no instrument shaft is coupled to the coupler and allowing selective positioning of the actuation shaft to the firing orientation when the instrument shaft is coupled to the coupler.

2. The handle assembly of claim 1, wherein the articulation mechanism comprises a release mechanism operable to disengage the articulation mechanism and reset the stapler jaw assembly to a longitudinally centered position relative to the instrument shaft.

3. The handle assembly of claim 2, wherein the articulation lockout mechanism engages the release mechanism of the articulation mechanism when no instrument shaft is coupled to the coupler.

4. The handle assembly of claim 1, wherein the articulation lockout mechanism comprises a lockout sleeve at the coupler of the handle body, and at least one lockout arm coupled to the lockout sleeve and extending proximally from the lockout sleeve to a proximal end, and a locking sleeve at the proximal end of the at least one lockout arm.

5. The handle assembly of claim 4, wherein the locking sleeve engages the articulation mechanism to prevent operation of the articulation mechanism with no instrument shaft coupled to the coupler.

6. The handle assembly of claim 4, wherein the at least one lockout arm comprises two lockout arms coupled to the lockout sleeve and each extending proximally from the lockout sleeve.

7. The handle assembly of claim 4, wherein the lockout sleeve, the at least one lockout arm, and the locking sleeve are biased distally with no instrument shaft coupled to the coupler.

8. The handle assembly of claim 7, wherein the lockout sleeve is moved proximally when the instrument shaft is coupled to the coupler.

9. The handle assembly of claim 4, further comprising a rotation mechanism configured to selectably position the actuation shaft in one of the firing orientation and the reverse orientation.

10. The handle assembly of claim 9, wherein the at least one lockout arm is engageable with the rotation mechanism to prevent rotation of the actuation shaft to the firing orientation when no instrument shaft is coupled to the coupler.

11. A handle assembly for a surgical stapler, the handle assembly comprising:
a handle body comprising a stationary handle and a movable handle pivotably coupled to the handle body; the handle body having a coupler configured to removably couple to an instrument shaft;
an actuation shaft mechanically coupled to the movable handle for manual actuation thereof, the actuation shaft slidable within the handle body along a longitudinal axis and rotatable relative to the longitudinal axis to selectively position the actuation shaft in one of a firing orientation and a reverse orientation;
a rotation mechanism to selectively rotate the actuation shaft between the firing orientation and the reverse orientation, the rotation mechanism comprising a gear rotatably fixed to the actuation shaft; and
a shaft coupling firing lockout mechanism, the shaft coupling firing lockout mechanism engageable with the gear to prevent rotation of the actuation shaft when no instrument shaft is coupled to the coupler.

12. The handle assembly of claim 11, wherein the shaft coupling firing lockout mechanism comprises at least one lockout arm comprising a lockout tab, the lockout tab engaging the gear when no instrument shaft is coupled to the coupler.

13. The handle assembly of claim 12, wherein the at least one lockout arm is movable when the instrument shaft is coupled to the coupler such that the lockout tab is disengaged from the gear.

14. The handle assembly of claim 12, wherein the gear comprises a slot formed therein, the lockout tab positionable in the slot to interfere with rotation of the gear.

15. The handle assembly of claim 12, wherein the shaft coupling firing lockout mechanism comprises a lockout sleeve positioned adjacent the coupler, the lockout sleeve coupled to the lockout arm.

16. The handle assembly of claim 15, wherein the lockout sleeve is longitudinally movable when the instrument shaft is coupled to the coupler.

17. The handle assembly of claim 12, wherein the lockout arm further comprises a second lockout tab protruding therefrom and a lockout recess between the lockout tab and the second lockout tab.
